(19) United States
(12) Patent Application Publication
(10) Pub. No.: US 2025/0311928 A1
(43) Pub. Date: Oct. 9, 2025

(54) SYSTEMS AND METHODS FOR PRESENTING DENTAL SCANS

(71) Applicant: Get-Grin Inc., Airmont, NY (US)

(72) Inventors: Alon Luis LIPNIK, Tel Aviv (IL); Pamela Sharon Oren-Artzi, Ausin, TX (US); Adam Benjamin Schulhof, New city, NY (US); Yarden Eilat-Bloch, Haifa (IL)

(21) Appl. No.: 19/244,402

(22) Filed: Jun. 20, 2025

Related U.S. Application Data
(63) Continuation of application No. PCT/US23/85455, filed on Dec. 21, 2023.
(60) Provisional application No. 63/434,478, filed on Dec. 22, 2022.

(51) Int. Cl. A61B 5/00; G06T 3/4038; G06T 7/00

(52) U.S. Cl. CPC A61B 5/0088; G06T 3/4038; G06T 7/0012; A61B 2503/06; G06T 2207/20221; G06T 2210/41

(57) ABSTRACT

The present disclosure provides methods and system for generating a panoramic intraoral presentation, comprising: providing intraoral video or images; analyzing the video or images; stitching the videos or images, and; presenting the panoramic intraoral presentation, wherein at least at least part of the gingival is visible.

### CROSS-REFERENCE TO RELATED APPLICATIONS

The present application is a continuation of International Patent Application PCT/US2023/085455, filed Dec. 21, 2023, which claims the benefit of U.S. Provisional Application Ser. No. 63/434,478, filed Dec. 22, 2022, each of which is expressly incorporated by reference herein in its entirety.

### TECHNICAL FIELD

The present invention, in at least some embodiments, relates to methods and system for dental assessment, and more specifically to methods and system for presenting dental scan.

### BACKGROUND

Dental professionals may treat and monitor a patient's dental condition based on in-person visits. Treatment and monitoring of a patient's dental condition may require a patient to schedule multiple in-person visits to a dentist. The quality of treatment and the accuracy of monitoring may vary depending on how often and how consistently a patient sees a dentist. In some cases, suboptimal treatment outcomes may result if a patient is unable or unwilling to schedule regular visits to a dentist.

### SUMMARY

Recognized herein is a need for dental monitoring solutions to allow dental assessment, optionally remote dental assessment, without requiring a dental professional to be physically present with the patient. Some dental professionals may use conventional teledentistry solutions to accommodate patients' needs and schedules. However, such conventional teledentistry solutions may provide inadequate levels of supervision. Further, such conventional teledentistry solutions may be limited by an inaccurate or insufficient monitoring of a patient's dental condition based on one or more photos taken by the patient, if the photos do not adequately capture various intraoral features.

The present disclosure provides methods and system for dental assessment, such as remote dental assessment. As used herein, the term “remote dental assessment” may refer to assessments conducted by remote personnel and may refer to the acquisition of one or more intraoral videos and/or intraoral images that can be assessed using a remote computing device, or can be sent to different remote locations for assessment. The methods and systems disclosed herein may provide a convenient solution and user experience for dental assessment presentation, in some embodiments, remote assessment, optionally enabling patients to capture one or more intraoral videos or images using a mobile device such as a smartphone. The methods and system disclosed herein, according to some embodiments, may provide dentists with a panoramic presentation of the patient's dental condition based on one or more dental scans captured remotely by the patient.

In an aspect, provided herein is a method for generating a panoramic intraoral presentation, comprising: (a) providing two or more intraoral videos or images of an intraoral region of a subject; (b) analyzing the two or more intraoral videos or images; and (c) stitching a first intraoral video or image of the two or more intraoral videos or images with a second intraoral video or image of the two or more intraoral videos or images, thereby generating the panoramic intraoral presentation, wherein the panoramic intraoral presentation comprises at least a portion of the first intraoral video or image and at least a portion of the second intraoral video or image, and wherein at least part of a gingival area of the subject is visible in the panoramic intraoral presentation.

In some cases, the two or more intraoral images or videos provided in (a) are acquired using a mobile digital camera and a hollow tubular adapter, wherein one opening of the hollow tubular adapter is attached to the mobile digital camera, and wherein the second opening of the hollow tubular adapter is configured to contact lips of the subject.

In some cases, the analyzing in (b) comprises selecting the first intraoral video or image and the second intraoral video or image. In some cases, the analyzing in (b) comprises identifying at least one point of similarity between the first intraoral video or image and the second intraoral video or image. In some cases, the analyzing in (b) comprises segmenting the two or more intraoral videos or images and/or designating one or more reference objects in the two or more intraoral videos or images. In some cases, the analyzing in (b) comprises identifying one or more teeth or gingival anomalies in the two or more intraoral video or images.

In some cases, the analyzing in (b) comprises comparing the two or more intraoral video or images to an additional image or video of the intraoral region in a different light spectrum than the two or more intraoral videos or images. In some cases, the first intraoral video or image is in a different light spectrum than the second intraoral video or image, and wherein the analyzing in (b) comprises comparing the first intraoral video or image to the second intraoral video or image. In some cases, the analyzing in (b) comprises comparing the two or more intraoral video or images to additional intraoral video or images previously taken of the intraoral region of the subject.

In some cases, the first intraoral video or image is taken prior to the second intraoral video or image, and wherein the analyzing in (b) comprises comparing the first intraoral video or image to the second intraoral video or image. In some cases, the analyzing in (b) comprises comparing the two or more intraoral videos or images to a prediction model. In some cases, the second intraoral video or image comprises a prediction model, and wherein the analyzing in (b) comprises comparing the first intraoral video or image to the prediction model.

In some cases, the analyzing in (b) comprises calibrating the two or more intraoral videos or images. In some cases, the panoramic intraoral representation generated in (c) comprises the at least one point of similarity. In some cases, the panoramic intraoral representation generated in (c) comprises (i) the segmented two or more intraoral videos or images or (ii) the one or more reference objects.

In some cases, the stitching in (c) comprises fitting the two or more intraoral videos or images to a template. In some cases, the template comprises a human dental layout. In some cases, the human dental layout comprises a forward-facing presentation of an upper arch and a lower arch in an aligned bite. In some cases, the human dental layout comprises a child dental layout. In some cases, the analyzing in (b) comprises fitting the two or more intraoral videos or images to at least one aligned arch. In some cases, the fitting the panoramic intraoral presentation to at least one aligned arch comprises straightening an arch of the two or more intraoral videos or images prior to the stitching in (c). In some cases, the stitching in (c) comprises using the one or more reference objects to generate the panoramic intraoral presentation.

In some cases, the method further comprises (d) presenting the panoramic intraoral presentation. In some cases, the method further comprises presenting the one or more teeth or gingival anomalies. In some cases, the method further comprises presenting the panoramic intraoral representation and the additional intraoral videos or images previously taken of the intraoral region of the subject. In some cases, the presenting in (d) comprises comparing the panoramic intraoral representation to a prediction model.

In some cases, the method further comprises: providing a previous panoramic intraoral presentation; comparing the panoramic intraoral presentation to the previous panoramic intraoral presentation; and presenting the comparison of the panoramic intraoral presentation and the previous panoramic intraoral presentation.

In some cases, the method further comprises presenting the panoramic intraoral presentation to a recipient. In some cases, the recipient is the subject. In some cases, the recipient is a health care personnel. In some cases, the panoramic intraoral presentation is converted into a dental chart of the user. In some cases, the panoramic intraoral presentation is converted into a score representing an oral hygienic condition of the user.

In some cases, the analyzing in (c) is done by a human. In some cases, the analyzing in (c) is done automatically by a computer software.

In another aspect, provided herein is a system for generating a panoramic intraoral image, comprising a non-transitory computer readable storage medium comprising instructions, the instructions configured to perform a method comprising: (a) providing two or more intraoral video or images of an intraoral region of a subject; (b) analyzing the two or more intraoral video or images; and (c) stitching a first intraoral video or image of the two or more intraoral videos or images with a second intraoral video or image of the two or more intraoral videos or images, thereby generating the panoramic intraoral image, wherein the panoramic intraoral presentation comprises at least a portion of the first intraoral video or image and at least a portion of the second intraoral video or image, and wherein at least part of a gingival area of the subject is visible in the panoramic intraoral presentation.

In some cases, the two or more intraoral images or videos provided in (a) are acquired using a mobile digital camera and a hollow tubular adapter, wherein one opening of the hollow tubular adapter is attached to the mobile digital camera, and wherein the second opening of the hollow tubular adapter is configured to contact lips of the subject. In some cases, the analyzing in (b) comprises selecting the first intraoral video or image and the second intraoral video or image. In some cases, the analyzing in (b) comprises identifying at least one point of similarity between the first intraoral video or image and the second intraoral video or image.

In some cases, the analyzing in (b) comprises segmenting the two or more intraoral videos or images and/or designating one or more reference objects in the two or more intraoral videos or images. In some cases, the analyzing in (b) comprises identifying one or more teeth or gingival anomalies in the two or more intraoral video or images. In some cases, the analyzing in (b) comprises comparing the two or more intraoral video or images to an additional image or video of the intraoral region in a different light spectrum than the two or more intraoral videos or images. In some cases, the first intraoral video or image is in a different light spectrum than the second intraoral video or image, and wherein the analyzing in (b) comprises comparing the first intraoral video or image to the second intraoral video or image.

In some cases, the analyzing in (b) comprises comparing the two or more intraoral video or images to additional intraoral video or images previously taken of the intraoral region of the subject. In some cases, the first intraoral video or image is taken prior to the second intraoral video or image, and wherein the analyzing in (b) comprises comparing the first intraoral video or image to the second intraoral video or image. In some cases, the analyzing in (b) comprises comparing the two or more intraoral videos or images to a prediction model.

In some cases, the second intraoral video or image comprises a prediction model, and wherein the analyzing in (b) comprises comparing the first intraoral video or image to the prediction model. In some cases, the analyzing in (b) comprises calibrating the two or more intraoral videos or images. In some cases, the panoramic intraoral representation generated in (c) comprises the at least one point of similarity. In some cases, the panoramic intraoral representation generated in (c) comprises (i) the segmented two or more intraoral videos or images or (ii) the one or more reference objects.

In some cases, the stitching in (c) comprises fitting the two or more intraoral videos or images to a template. In some cases, the template comprises a human dental layout. In some cases, the human dental layout comprises a forward facing presentation of an upper arch and a lower arch in an aligned bite. In some cases, the human dental layout comprises a child dental layout. In some cases, the stitching in (c) comprises fitting the panoramic intraoral presentation to at least one aligned arch. In some cases, the fitting the panoramic intraoral presentation to at least one aligned arch comprises straightening an arch of the two or more intraoral videos or images prior to the stitching in (c).

In some cases, the stitching in (c) comprises using the one or more reference objects to generate the panoramic intraoral presentation. In some cases, the method further comprises (d) presenting the panoramic intraoral presentation. In some cases, the method further comprises presenting the one or more teeth or gingival anomalies. In some cases, the method further comprises presenting the panoramic intraoral representation and the additional intraoral videos or images previously taken of the intraoral region of the subject.

In some cases, the presenting in (d) comprises comparing the panoramic intraoral representation to a prediction model. In some cases, the method further comprises providing a previous panoramic intraoral presentation; comparing the panoramic intraoral presentation to the previous panoramic intraoral presentation; and presenting the comparison of the panoramic intraoral presentation and the previous panoramic intraoral presentation.

In some cases, the method further comprises presenting the panoramic intraoral presentation to a recipient. In some cases, the recipient is the subject. In some cases, the recipient is a health care personnel. In some cases, the panoramic intraoral presentation is converted into a dental chart of the user. In some cases, the panoramic intraoral presentation is converted into a score representing an oral hygienic condition of the user. In some cases, the analyzing in (c) is done by a human. In some cases, the analyzing in (c) is done automatically by a computer software.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system of the present invention involves performing or completing selected tasks or steps manually, automatically, or a combination thereof. Moreover, according to actual instrumentation and equipment of preferred embodiments of methods, apparatus and systems of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, devices or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a flow-chart schematically illustrating a method for a panoramic intraoral presentation, in accordance with an embodiment described herein.

FIG. 2 is a schematically illustrates three or more intraoral video frames or intraoral images stitched together to form a panoramic intraoral representation, in accordance with an embodiment described herein.

FIG. 3 is a schematically illustration of an intraoral video frame or image with one or more oral landmarks or anomalies identified and labeled, in accordance with an embodiment described herein.

FIG. 4 schematically illustrates a computer system that is programmed or otherwise configured to implement at least some of the methods disclosed herein, in accordance with an embodiment described here.

FIG. 5 is a flow-chart schematically illustrating a method of presenting a summary of a dental scan comprising a panoramic representation, in accordance with an embodiment described here.

FIG. 6A and FIG. 6B are show examples of a tooth template that can be used to assemble a panoramic presentation, in accordance with an embodiment described here.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

The term “intraoral adapter,” as used herein, generally refers to an object or device that can at least partly be set on the intraoral region of the mouth.

The term “dental scan,” as used herein, generally refers to a digital representation of the interior part of the mouth, usually the dental scan is an image or a video of at least part of an arch and including at least part of a teeth.

The terms “UV, visible light, IR”, as used herein, generally refers to sections in the electromagnetic spectrum. UV refers to ultraviolet light with wavelength in the range of 100 to 400 nm. Visible refers to light with a wavelength in the range of 100 to 700 nm (the part of the electromagnetic spectrum a human can see). IR refers to infrared light with a wavelength above 700 nm.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

### Overview

According to an aspect, the present disclosure provides methods and systems for intraoral assessment. As used herein, “intraoral assessment” may refer to assessment of an appearance or condition of an intraoral region of a subject. The methods and system disclosed herein may provide a convenient one presentation of an intraoral assessment such as panoramic intraoral image that include all aspects acquired by the dental scan. The methods and systems disclosed herein may provide dentists and orthodontists or any health care personal with a detailed analysis of the patient's dental condition based on a video or on one or more images of at least one tooth, such as one or more teeth, one or more dental arches or both dental arches of a subject, in some embodiments captured remotely by a user, In some embodiments, the user is the subject. In some embodiments the user is a person other than the subject, present at the same location as the subject and assessment of the captured video or image(s) is performed at a remote location.

As used herein the term “dental scope” or “dental adaptor” refer to a device that can be attached to a camera or mobile phone and enables capturing at least one intraoral image or videos of a subject, the term “dental scope” or “dental adaptor” may refer to a hollow tubular adapter wherein one opening of the hollow tubular adapter is attached to the mobile digital camera and the second opening of the hollow tubular adapter was in contact with the user inner lips and the lips lay on the outer of the second opening of the hollow tubular adapter. An example and possible uses can be seen in U.S. patent application Ser. No. 17/336,997.

As used herein, the term “dental scan” refers to intraoral videos or intraoral images that are the results of using the “dental scope” or “dental adaptor” as attached to a digital camera such as mobile device while acquiring an intraoral video or intraoral image. An example for dental scan, and possible uses, can be seen in U.S. patent application Ser. No. 17/336,997. The intraoral videos or intraoral images or the dental scan provided to the method and systems of the present disclosure may be implemented using a software application that is configured to enable a dental patient to capture videos and/or images of intraoral regions. The software application may be used by a user or a subject (e.g., a dental patient) in conjunction with a mobile device to remotely monitor a dental condition of the subject. The dental condition may comprise a development, a growth, a movement, an appearance, a condition, a physical arrangement, a position, and/or an orientation of the subject's teeth or/and the subject's soft tissues in the oral cavity.

As used herein. “remote monitoring” may refer to monitoring the intraoral region of a subject and/or a condition of an intraoral region of a subject, wherein the monitoring is performed at one or more locations remote from the subject. For example, a dentist or a medical specialist may monitor the intraoral anatomy or intraoral condition at a first location that is different than a second location at which the subject is located. The first location and the second location may be separated by a distance spanning at least 1 meter, 1 kilometer, 10 kilometers, 100 kilometers, 1000 kilometers, or more.

The remote monitoring may be performed by assessing an intraoral condition of the subject using one or more dental scans captured from the subject when the subject is located remotely from the dentist or a dental office. In some cases, the remote monitoring may be performed in real-time such that a dentist is able to assess the dental condition when a subject uses a mobile device to acquire one or more videos or images of one or more intraoral regions in the patient's mouth. The remote monitoring may be performed using equipment, hardware, and/or software that is not physically located at a dental office.

The software application for dental assessment may be configured to run on a mobile device. The mobile device may comprise a smartphone, a tablet, a laptop, or any suitable device that may be used by a patient to capture one or more dental assessments. The software application may be installed on a mobile device of a user. The software application may be a patient-side software application. Alternatively, the software application for dental assessment may be configured to run on a fixed-location device, such as a desktop computer.

As used herein, the terms “panoramic intraoral presentation”, “panoramic intraoral image” and “panoramic representation” refers to an image showing at least part of a dental arch of teeth of a subject, made from at least two intraoral video frames or intraoral images that contain at least one tangential or overlapping area.

### Method for Panoramic Intraoral Presentation

Another aspect described herein is a method for a panoramic intraoral presentation, comprising: providing intraoral video or images; analyzing the video or images; stitching the images; and presenting the panoramic intraoral presentation. In some cases, at least at least part of the gingival is visible in the panoramic intraoral presentation.

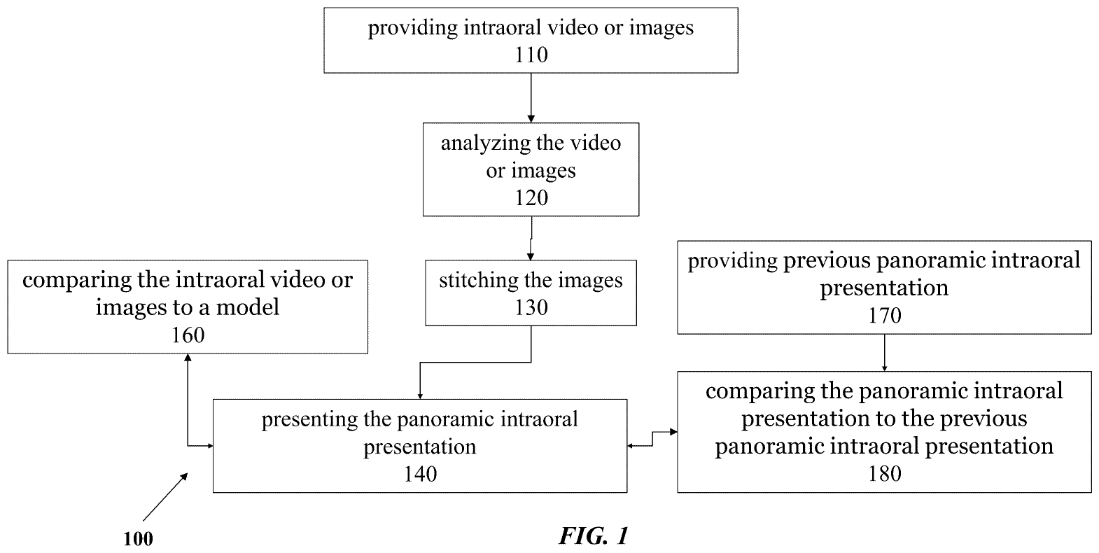

FIG. 1 schematically illustrates a method 100 for generating a panoramic intraoral presentation, in accordance with systems and methods described herein. In some cases, the method includes providing intraoral video or images 110. The digital files can be in any format that represent video or images. In some cases, more than one intraoral videos or images are provided. For example, 2, 3, 4, 5, or 6 intraoral videos or images are provided.

According to some embodiments, the method is a computer implemented.

According to some embodiments, the provided intraoral images or videos were acquired using a mobile digital camera and a hollow tubular adapter. A first opening of the hollow tubular adapter can be attached to the mobile digital camera and a second opening of the hollow tubular adapter can be in contact with lips of a user. The user's inner lips may contact the outer of the second opening of the hollow tubular adapter. According to some embodiments the mobile digital camera is a mobile phone including a camera. In some cases, the mobile phone is a smartphone.

The method may further include analyzing the intraoral video or images 120. The analyzing may be conducted manually or by a computer algorithm. According to some embodiments, the analyzing of the intraoral video comprises selection of at least two video frames. According to some embodiments, the selected video frame includes appearance of all the desired teeth. The desired teeth may include all the teeth of a user. In some cases, the analyzing of the intraoral images comprises selection of at least two images. According to some embodiments, the selected images include appearance of all the desired teeth. In some cases, the desired teeth include all the teeth of a user.

According to some embodiments, the analyzing of the intraoral video or images 120 comprises identifying candidate points of similarity. The candidate points of similarity can appear in more than one video frame or image. The candidate points of similarity can share the same shape, the same color, the same morphologic area, or the same relative location in the intraoral region.

According to some embodiments, analyzing the intraoral video or images 120 comprises the segmentation and/or the designation of objects in the intraoral video or images.

According to some embodiments, the analyzing of the intraoral video or images 120 comprises identifying one or more teeth or gingival anomalies in the intraoral video or images. According to some embodiments, the anomaly is a movement of an object, a color, a porosity, a light reflection, a light absorbent, a leak, an addition, or abnormal appearance.

According to some embodiments, analyzing the intraoral video or images comprises comparing the intraoral video or images to an additional image or video of the intraoral region in a different light spectrum than the two or more intraoral videos or images. According to some embodiments, the analyzing of the intraoral video or images comprises comparing the intraoral video or images to previous intraoral video or images taken at an earlier time period. In some cases, the previous intraoral video or images were taken before a user undergoes dental or orthodontic treatment. According to some embodiments, the analyzing the intraoral video or images comprises comparing the intraoral video or images to a prediction model.

According to some embodiments, the analyzing the intraoral video or images comprises steps of calibrating the intraoral video or images. According to some embodiments, the calibration comprises color matching of objects in the intraoral video or images. According to some embodiments, the calibration comprises adjusting the lighting of objects in the intraoral video or images. According to some embodiments, the calibration comprises size adjustment of objects in the intraoral video or images.

According to some embodiments, the analyzing of the intraoral video or images 120 is based on the results of a machine learning algorithm.

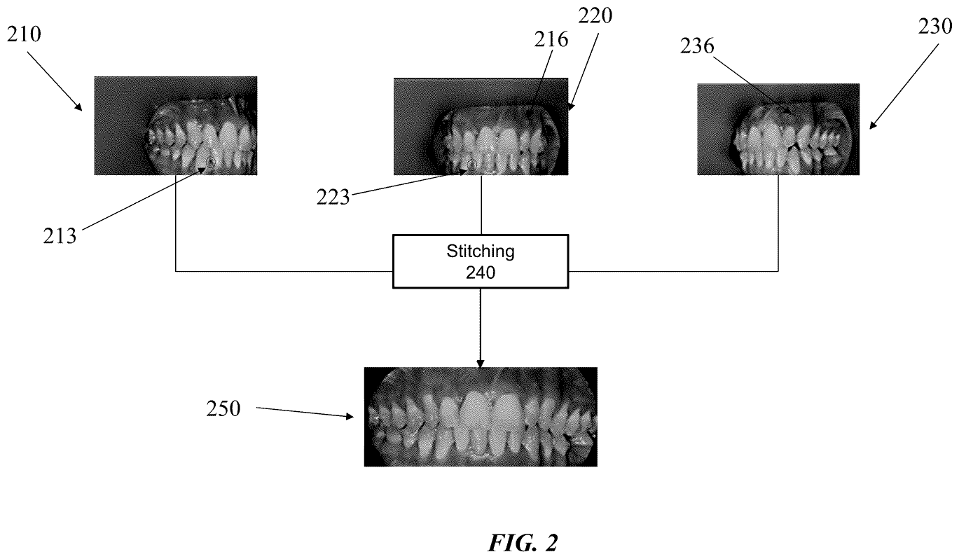

The methods described herein can include stitching the intraoral video frames or images 130 into a panoramic intraoral presentation, FIG. 2 shows an example of a possible stitching. In FIG. 2, three intraoral video frames or images, right-side 210, front 220, and left-side 230 with the respectively identified point of similarity 213, 223 and 216, 236 are stitched 240) according to the identified point of similarity 213, 223 and 216, 236 into a panoramic intraoral presentation 250.

According to some embodiments, stitching the intraoral video frames or images into a panoramic intraoral presentation comprises at least one point of similarity. According to some embodiments, the stitching the intraoral video frames or images into a panoramic intraoral presentation comprises at least one segmented area and/or designated objects.

According to some embodiments, stitching the intraoral video frames or images into a panoramic intraoral presentation comprises using random sample consensus (RANSEC). According to some embodiments, stitching the intraoral video frames or images into a panoramic intraoral presentation comprises using Pyramid generation. According to some embodiments, the Pyramid generation is a Laplacian pyramid. According to some embodiments, stitching the intraoral video frames or images into a panoramic intraoral presentation comprises using different projective layout as Rectilinear projection, Cylindrical projection, Spherical projection, or other projections.

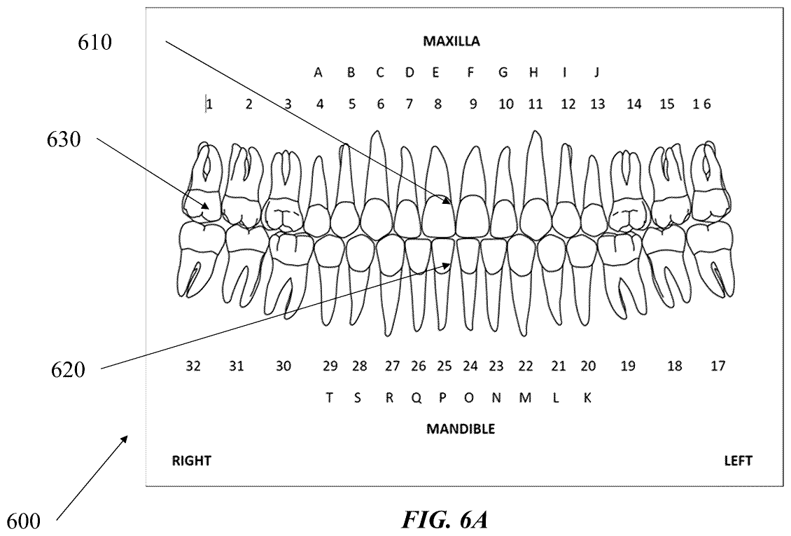

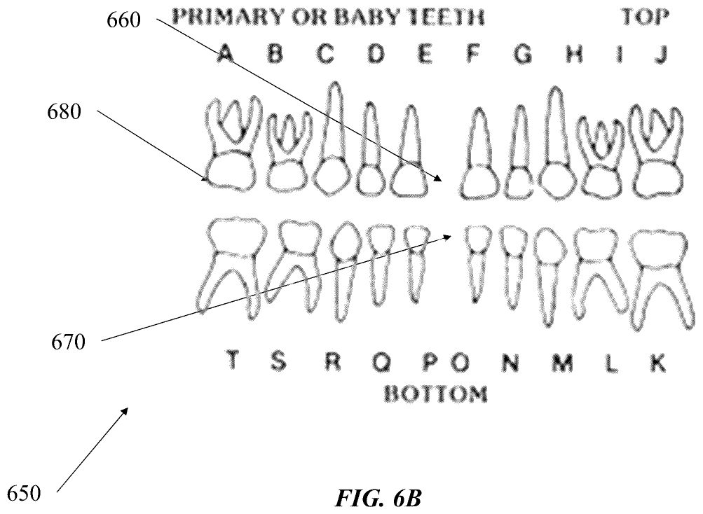

According to some embodiments, stitching the intraoral video frames or images 130 into a panoramic intraoral presentation comprises fitting the panoramic intraoral to a template. According to some embodiment the template is a human dental layout. FIG. 6A shows an example of a human dental layout 600 with adult human teeth forward facing presentation of the upper arch (maxilla 610) and the lower arch (mandible 620) in an aligned bite. According to some embodiments, the fitting of the intraoral video frames or images 130 to the template is directed to the crown section 630 of each tooth or group of teeth. According to some embodiments, the human dental layout can be a layout of a child, an adult, or a human at any other grown stage. FIG. 6B shows an example of a human dental layout 650 with child human teeth forward facing presentation of the upper arch (maxilla 660) and the lower arch (mandible 670) in an aligned bite. According to some embodiments, the fitting of the intraoral video frames or images 130 to a template is directed to the crown section 680 of each tooth or group of teeth. According to some embodiments, the stitching the intraoral video frames or images into a panoramic intraoral presentation comprises fitting the panoramic intraoral to at least one aligned arch. According to some embodiments, the stitching of the intraoral video frames or images into a panoramic intraoral presentation comprises fitting the panoramic intraoral to at least one point of interest.

According to some embodiments, the presenting of the panoramic intraoral presentation 140 is done by generating digital file and presenting the digital file on a screen. The screen may be a screen at remote location. The presentation may comprise transferring the digital file to the remote location. According to some embodiments, the presenting comprises printing the panoramic intraoral presentation.

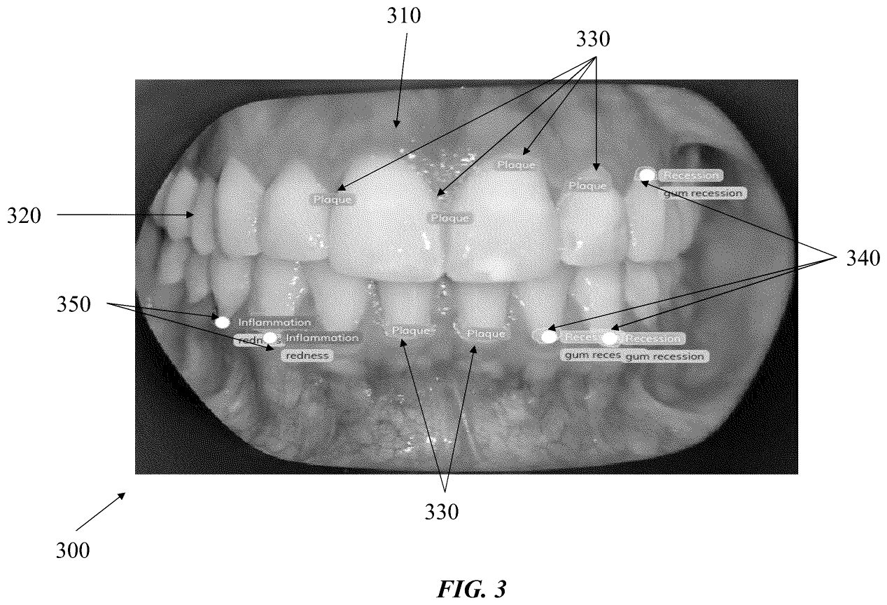

According to some embodiments, the presenting of the panoramic intraoral presentation 140 in FIG. 1 comprises pointing to one or more teeth or gingival anomalies. FIG. 3 shows an example of pointing to one or more teeth or gingival anomalies in an intraoral photo 300. The gingival are 310 and the teeth 320 are distinctly shown. Anomalies such as plaque 330, gum recession 340, redness of inflammations 350, and others not shown here can be presented. According to some embodiments, identification of the one or more anomalies is conducted by reviewing the intraoral images or video. This review can be done automatically by a computer according to preset properties. In some cases, an identified anomaly is on a different layer of the image. For example, the identified anomaly may not be part of the dental image or video but may be overlaid on top. In some cases, an identified anomaly is presented separately from the original dental image or video.

According to some embodiments, the presenting of the panoramic intraoral presentation 140 comprises a result of comparing 180 the panoramic intraoral presentation to a previous panoramic intraoral presentation 170.

According to some embodiments, the presenting of the panoramic intraoral presentation 140 comprises comparing the intraoral video or images to a prediction model 160. According to some embodiments, the prediction model 160 can be a speculative result of prediction due to orthodontic involvement, dental treatment outcome, previous condition, human grown result, traumas, illness, or any unexpected cases.

According to some embodiments, the method further comprises: providing a previous panoramic intraoral presentation 170; comparing the panoramic intraoral presentation to the previous panoramic intraoral presentation 180; and presenting the panoramic intraoral presentation 140, which comprises a result of comparing the panoramic intraoral presentation to the previous panoramic intraoral presentation.

### Systems for Generating and Displaying a Panoramic Intraoral Image

Another aspect provided herein is a system for panoramic intraoral image, comprising: a processor; a storage element connected to the processor; encoded instruction stored in the storage element, wherein the encoded instructions when implemented by the processor, configure the system to: receive intraoral video or images; analyze the intraoral video or images; and stitch the intraoral video or images into panoramic intraoral image; wherein at least at least part of the gingival is visible in the panoramic intraoral image.

According to some embodiments, the provided intraoral images or videos were acquired using a mobile digital camera and a hollow tubular adapter. A first opening of the hollow tubular adapter can be attached to the mobile digital camera and a second opening of the hollow tubular adapter can be in contact with lips of a user. The user's inner lips may contact the outer of the second opening of the hollow tubular adapter. According to some embodiments the hollow tubular adapter is similar to the hollow tubular adapter disclosed in U.S. patent application Ser. No. 17/336,997, or others.

According to some embodiments, the analyzing of the intraoral video comprises selection of at least two video frames. According to some embodiments, the selected frames comprise an object or mark that appear in at least two frames or images.

According to some embodiments, the analyzing of the intraoral video or images comprises identifying candidate points of similarity. The candidate points of similarity can appear in more than one video frame or image. The candidate points of similarity can share the same shape, the same color, the same morphologic area, or the same relative location in the intraoral region.

According to some embodiments, analyzing the intraoral video or images comprises the segmentation and/or the designation of objects in the intraoral video ir images.

According to some embodiments, the analyzing of the intraoral video or images comprises identifying one or more teeth or gingival anomalies in the intraoral video or images. According to some embodiments, the anomaly is a movement of an object, a color, a porosity, a light reflection, a light absorbent, a leak, an addition, or abnormal appearance.

According to some embodiments, analyzing the intraoral video or images comprises comparing the intraoral video or images to an additional image or video of the intraoral region in a different light spectrum than the two or more intraoral videos or images. According to some embodiments, the analyzing of the intraoral video or images comprises comparing the intraoral video or images to previous intraoral video or images taken at an earlier time period. In some cases, the previous intraoral video or images were taken before a user undergoes dental or orthodontic treatment. According to some embodiments, the analyzing the intraoral video or images comprises comparing the intraoral video or images to a prediction model.

According to some embodiments, the analyzing the intraoral video or images comprises steps of calibrating the intraoral video or images. According to some embodiments, the calibration comprises color matching of objects in the intraoral video or images. According to some embodiments, the calibration comprises adjusting the lighting of objects in the intraoral video or images. According to some embodiments, the calibration comprises size adjustment of objects in the intraoral video or images.

According to some embodiments, the stitching intraoral video or images into a panoramic intraoral image comprises at least one point of similarity.

According to some embodiments, the stitching intraoral video or images into a panoramic intraoral image comprises at least one segmented area and/or designated objects.

According to some embodiments, stitching intraoral video or images into a panoramic intraoral image comprises fitting the panoramic intraoral to a to a template. According to some embodiment the template is a human dental layout.

FIG. 6A shows an example of a human dental layout 600 with adult human teeth forward facing presentation of the upper arch (maxilla 610) and the lower arch (mandible 620) in an aligned bite. According to some embodiments, the fitting of the intraoral video frames or images 130 to the template is directed to the crown section 630 of each tooth or group of teeth. According to some embodiments, the human dental layout can be a layout of a child, an adult, or a human at any other grown stage. FIG. 6B shows an example of a human dental layout 650 with child human teeth forward facing presentation of the upper arch (maxilla 660) and the lower arch (mandible 670) in an aligned bite. According to some embodiments, the fitting of the intraoral video frames or images 130 to a template is directed to the crown section 680 of each tooth or group of teeth. According to some embodiments, the stitching the intraoral video frames or images into a panoramic intraoral presentation comprises fitting the panoramic intraoral to at least one aligned arch. According to some embodiments, the stitching of the intraoral video frames or images into a panoramic intraoral presentation comprises fitting the panoramic intraoral to at least one point of interest.

According to some embodiments, the system further comprises transferring the panoramic intraoral image to a recipient.

According to some embodiments, the panoramic intraoral image comprises identifying one or more teeth or gingival anomalies.

According to some embodiments, the panoramic intraoral image comprises a result of comparing the intraoral video or images to previous intraoral video or images.

According to some embodiments, the panoramic intraoral image comprises a result of comparing the intraoral video or images to a prediction model.

According to some embodiments, the recipient is a health care personal or a user.

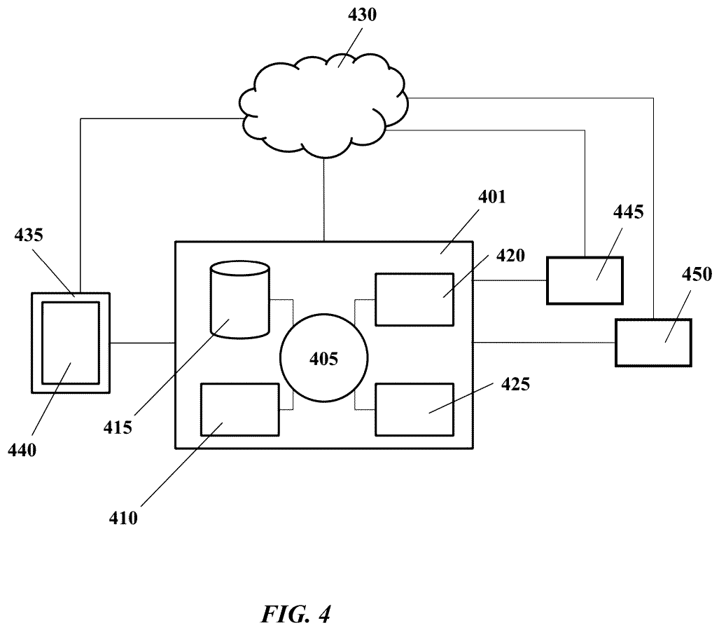

FIG. 4 shows a computer system 401 that is programmed or otherwise configured to implement a method for panoramic intraoral image. The computer system 401 may be configured to, for example, process intraoral videos or images captured using the camera of the mobile device, analyze the videos or images, and stitching the intraoral video or images into panoramic intraoral image. The computer system 401 can be an electronic device of a user or a computer system that is remotely located with respect to the electronic device, or even in the cloud. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk, Solid State drive or equivalent storage unit), communication interface 420 (e.g., network adaptor) for communicating with one or more other systems, and peripheral devices 425, such as cache, other memory, data storage and/or electronic display adaptors. The memory 410, storage unit 415, interface 420) and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental patient, or a dentist). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods and systems as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a dental patient to view one or more intraoral images or videos captured using a mobile device of the subject or the dental patient. In some cases, the electronic display 435 may be the feedback element providing the generated output, for example displaying message or shape or light in accordance to some embodiments. The portal may be provided through an application programming interface (API). A user or entity can also interact with various elements in the portal via the UI. Examples of UI's include, without limitation, a graphical user interface (GUI) and web-based user interface.

The computer system 401 can include or be in communication with a camera 445 for providing, for example, ability to capture videos or images of the intraoral of a user or a dental patient.

Methods and systems of the present disclosure can be implemented by way of one or more algorithms. An algorithm can be implemented by way of software upon execution by the central processing unit 405. The algorithm can, for example, implement a method for dental scan. The method may comprise processing videos or images captured using the camera of the mobile device or processing dental assessment data sensed by at least one sensor that can be used to analyze and compare to at least one dental assessment properties and executed to generate output.

### Systems and Methods for Presenting a Summary of a Dental Scan

Another aspect described herein is a method to present a summary of a dental scan, comprising: providing a dental scan video or dental scan images; processing the dental scan video or dental scan images; stitching the dental scan video or dental scan images into a panoramic representation. In some cases, the panoramic representation comprises at least two parts of dental scan images or dental scan video frames, and at least part of the gingival.

According to some embodiments, the summary of a dental scan includes presenting a finding of one or more anomalies. According to some embodiments, an anomaly is identified by reviewing the intraoral images or video. The reviewing of the intraoral images or video can be done automatically by the computer according to preset properties. In some cases, the identified anomaly is on different layer. In some cases, the identified anomaly can be presented separately.

According to some embodiments, the anomaly comprises tooth appearance. The tooth appearance may include: plaque, tartar, inflammation, recession, caries, gum recession or abnormality, wear, malocclusion, stains, cracks, filling, or crowns, or any combination thereof, and more.

According to some embodiments, the anomaly comprises soft tissue appearance. The soft tissue appearance may include; color, ulcer, infection, dryness, gum recession, gum abnormality growth, or inflammation, or a combination thereof, and more.

According to some embodiments, the provided intraoral images or videos were acquired using a mobile digital camera and a hollow tubular adapter. A first opening of the hollow tubular adapter can be attached to the mobile digital camera and a second opening of the hollow tubular adapter can be in contact with lips of a user. The user's inner lips may contact the outer of the second opening of the hollow tubular adapter.

According to some embodiments, the processing is done by human.

According to some embodiments, the processing is done automatically by a computer software. According to some embodiments the computer software outcome is based on results of machine learning algorithms that was trained for the required processing.

According to some embodiments, the processing comprises selection of at least two video frames from the dental scan video.

According to some embodiments, the processing comprises identifying candidate points of similarity and/or segmentation and/or designated objects.

According to some embodiments, the processing comprises identifying one or more teeth or gingival anomolies in the dental scan video or dental scan images. According to some embodiments the anomaly is a movement of an object, a color, a porosity, a light reflection, a light absorbent, a leak, an addition, or abnormal appearance. According to some embodiments, the analyzing of the intraoral video or images comprises comparing the intraoral video or images to additional intraoral video or images taken in a different light spectrum.

According to some embodiments, the processing comprises comparing the dental scan video or dental scan images to additional dental scan video or images taken in a different light spectrum intraoral dental scan video or dental scan images.

According to some embodiments, the processing comprises comparing the dental scan video or dental scan images to previous dental scan video or dental scan images.

According to some embodiments, the processing comprises comparing the dental scan video or dental scan images to a model or a template.

According to some embodiments, the analyzing the intraoral video or images comprises steps of calibrating the intraoral video or images. According to some embodiments, the calibration comprises color matching of objects in the intraoral video or images. According to some embodiments, the calibration comprises adjusting the lighting of objects in the intraoral video or images. According to some embodiments, the calibration comprises size adjustment of objects in the intraoral video or images.

According to some embodiments, the stitching comprises identifying at least one point of similarity.

According to some embodiments, the stitching comprises at least one segmented area and/or designated objects.

According to some embodiments, stitching the intraoral video frames or images 130 into a panoramic intraoral presentation comprises fitting the panoramic intraoral to a template. According to some embodiment the template is a human dental layout. FIG. 6A shows an example of a human dental layout 600 with adult human teeth forward facing presentation of the upper arch (maxilla 610) and the lower arch (mandible 620) in an aligned bite. According to some embodiments, the fitting of the intraoral video frames or images 130 to the template is directed to the crown section 630 of each tooth or group of teeth. According to some embodiments, the human dental layout can be a layout of a child, an adult, or a human at any other grown stage. FIG. 6B shows an example of a human dental layout 650 with child human teeth forward facing presentation of the upper arch (maxilla 660) and the lower arch (mandible 670) in an aligned bite.

According to some embodiments, the stitching comprises fitting the panoramic intraoral to at least one aligned arch.

According to some embodiments, the panoramic intraoral presentation is a digital image and is delivered to at least one receiver.

According to some embodiments, the receiver is a dental specialist.

According to some embodiments, the receiver is a user.

According to some embodiments, the panoramic representation is converted into a dental chart of a user. The term dental chart, as generally used herein, refers to a table that presents the status of the teeth as used in dental clinics.

According to some embodiments, the panoramic representation is converted into a score representing a user's oral hygienic condition. According to some embodiments, each parameter of the tooth receives a score that represent the tooth status for this parameter. The scores can be added and/or averaged to get the user score.

According to some embodiments, the panoramic representation is converted into a score representing a user's oral health condition. According to some embodiments, each parameter of the tooth receives a score that represent the tooth status for this parameter. The scores can be added and/or averaged to get the user score.

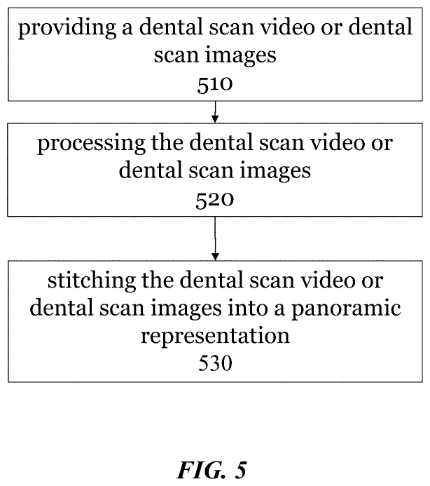

FIG. 5 is a flow-chart schematically illustrating a method for presenting a summary of a dental scan, in accordance with the principles described herein.

Method 500 comprises providing a dental scan video or dental scan images 510. The provided dental scan video or dental scan images can be acquired by using a mobile digital camera and a hollow tubular adapter, wherein one opening of the hollow tubular adapter is attached to the mobile digital camera and the second opening of the hollow tubular adapter is in contact with the user inner lips and the lips lay on the outer of the second opening of the hollow tubular adapter or from a storage drive. The provided dental scan video or dental scan images can then go to the step of processing the dental scan video or dental scan images 520. In according to the embodiments above, the processing among other things can provide at least two points of similarity. In the next step, the provided dental scan video or dental scan images can be stitched into a panoramic representation 530.

While embodiments have been shown and described herein, it will be obvious to those skilled in the art that such embodiments are provided by way of example only. It is not intended that the systems and methods described herein be limited by the specific examples provided within the specification. While the systems and methods described herein has been described with reference to the aforementioned specification, the descriptions and illustrations of the embodiments herein are not meant to be construed in a limiting sense.

Numerous variations, changes, and substitutions will now occur to those skilled in the art without departing from the systems and methods described herein. Furthermore, it shall be understood that all aspects of the systems and methods described herein are not limited to the specific depictions, configurations or relative proportions set forth herein which depend upon a variety of conditions and variables. It should be understood that various alternatives to the embodiments described herein may be employed in practicing the systems and methods described herein. It is therefore contemplated that the systems and methods described herein shall also cover any such alternatives, modifications, variations or equivalents. It is intended that the following claims define the scope of the systems and methods described herein and that methods and structures within the scope of these claims and their equivalents be covered thereby.

What is claimed is:

1. A method for generating a panoramic intraoral presentation, comprising:
(a) providing two or more intraoral videos or images of an intraoral region of a subject:
(b) analyzing the two or more intraoral videos or images; and
(c) stitching a first intraoral video or image of the two or more intraoral videos or images with a second intraoral video or image of the two or more intraoral videos or images, thereby generating the panoramic intraoral presentation, wherein the panoramic intraoral presentation comprises at least a portion of the first intraoral video or image and at least a portion of the second intraoral video or image, and wherein at least part of a gingival area of the subject is visible in the panoramic intraoral presentation,
wherein the two or more intraoral images or videos provided in (a) are acquired using a mobile digital camera and a hollow tubular adapter comprising two or more openings, wherein a first opening of the hollow tubular adapter is attached to the mobile digital camera, and wherein a second opening of the hollow tubular adapter is configured to contact lips of the subject.

2. The method of claim 1, wherein the analyzing in (b) comprises identifying at least one point of similarity between the first intraoral video or image and the second intraoral video or image.

3. The method of claim 1, wherein the analyzing in (b) comprises segmenting the two or more intraoral videos or images and/or designating one or more reference objects in the two or more intraoral videos or images.

4. The method of claim 1, wherein the analyzing in (b) comprises identifying one or more teeth or gingival anomalies in the two or more intraoral video or images.

5. The method of claim 1, wherein the analyzing in (b) comprises comparing the two or more intraoral video or images to an additional image or video of the intraoral region in a different light spectrum than the two or more intraoral videos or images.

6. The method of claim 1, wherein the analyzing in (b) comprises comparing the two or more intraoral videos or images to a prediction model.

7. The method of claim 1, wherein the analyzing in (b) comprises calibrating the two or more intraoral videos or images.

8. The method of claim 1, wherein the stitching in (c) comprises fitting the two or more intraoral videos or images to a template, wherein the template comprises a human dental layout.

9. The method of claim 1, further comprising (d) presenting the panoramic intraoral presentation.

10. The method of claim 1, wherein the panoramic intraoral presentation is converted into a dental chart of the user.

11. A system for generating a panoramic intraoral image, comprising a non-transitory computer readable storage medium comprising instructions, the instructions configured to perform a method comprising:
(a) providing two or more intraoral videos or images of an intraoral region of a subject:
(b) analyzing the two or more intraoral video or images; and
(c) stitching a first intraoral video or image of the two or more intraoral videos or images with a second intraoral video or image of the two or more intraoral videos or images, thereby generating the panoramic intraoral image, wherein the panoramic intraoral presentation comprises at least a portion of the first intraoral video or image and at least a portion of the second intraoral video or image, and wherein at least part of a gingival area of the subject is visible in the panoramic intraoral presentation,
wherein the two or more intraoral images or videos provided in (a) are acquired using a mobile digital camera and a hollow tubular adapter comprising two or more openings, wherein a first opening of the hollow tubular adapter is attached to the mobile digital camera, and wherein a second opening of the hollow tubular adapter is configured to contact lips of the subject.

12. The system of claim 11, wherein the analyzing in (b) comprises identifying at least one point of similarity between the first intraoral video or image and the second intraoral video or image.

13. The system of claim 11, wherein the analyzing in (b) comprises segmenting the two or more intraoral videos or images and/or designating one or more reference objects in the two or more intraoral videos or images.

14. The system of claim 11, wherein the analyzing in (b) comprises identifying one or more teeth or gingival anomalies in the two or more intraoral video or images.

15. The system of claim 11, wherein the analyzing in (b) comprises comparing the two or more intraoral video or images to an additional image or video of the intraoral region in a different light spectrum than the two or more intraoral videos or images.

16. The system of claim 11, wherein the analyzing in (b) comprises comparing the two or more intraoral videos or images to a prediction model.

17. The system of claim 11, wherein the analyzing in (b) comprises calibrating the two or more intraoral videos or images.

18. The system of claim 11, wherein the stitching in (c) comprises fitting the two or more intraoral videos or images to a template, wherein the template comprises a human dental layout.

19. The system of claim 18, wherein the human dental layout comprises a child dental layout.

20. The system of claim 11, wherein the stitching in (c) comprises fitting the panoramic intraoral presentation to at least one aligned arch.

21. The system of claim 20, wherein the fitting the panoramic intraoral presentation to at least one aligned arch comprises straightening an arch of the two or more intraoral videos or images prior to the stitching in (c).

22. The system of claim 11, further comprising (d) presenting the panoramic intraoral presentation.

23. The system of claim 11, wherein the panoramic intraoral presentation is converted into a dental chart of the user including a score representing an oral hygienic condition of the user.
